(19) United States
(12) Patent Application Publication
(10) Pub. No.: US 2022/0235034 A1
(43) Pub. Date: Jul. 28, 2022

(54) SELECTIVE HISTAMINE H3 RECEPTOR ANTAGONISTS FOR TREATING AUTISM SPECTRUM DISORDER

(71) Applicant: RICHTER GEDEON NYRT., Budapest (HU)

(72) Inventors: Viktor ROMÁN, Érd (HU); György István LÉVAY, Budakeszi (HU)

(21) Appl. No.: 17/614,412

(22) PCT Filed: May 29, 2020
(86) PCT No.: PCT/IB2020/055104

§ 371 Date: Nov. 26, 2021

(30) Foreign Application Priority Data
May 31, 2019 (HU) P1900190

(51) Int. Cl. C07D 401/14; A61P 25/18; A61K 45/06

(52) U.S. Cl. CPC C07D 401/14; A61K 45/06; A61P 25/18

(57) ABSTRACT

The present invention relates to the selective histamine H3 receptor antagonists/inverse agonists 1-[4-(4-{3-[(2R)-2-methylpyrrolidin-1-yl]-propoxy}-phenoxy)-piperidin-1-yl]-ethan-1-on, its pharmaceutically acceptable salts, derivatives, pharmaceutical compositions, active metabolites and combinations for use in the treatment of symptoms of autism spectrum disorder (ASD).

### FIELD OF THE INVENTION

The present invention relates to the selective histamine H3 receptor antagonists/inverse agonists 1-[4-(4-{3-[(2R)-2-methylpyrrolidin-1-yl]-propoxy}-phenoxy)-piperidin-1-yl]-ethan-1-on, their pharmaceutically acceptable salts, derivatives, pharmaceutical compositions, active metabolites and combinations for use in the treatment of symptoms of autism spectrum disorder (ASD).

### BACKGROUND OF THE INVENTION

Patent application WO 2014/136075 A1 discloses phenoxypiperazine derivatives, a process for their preparation and their therapeutic applications for the treatment of conditions that require modulation of histamine H3 receptors. According to WO 2014/136075A1 1-[4-(4-{3-[(2R)-2-methylpyrrolidin-1-yl]-propoxy}-phenoxy)-piperidin-1-yl]-ethan-1-on corresponding to Formula 1

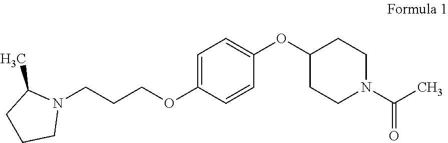 [SMILES: CC(=O)N1CCC(Oc2ccc(OCCCN3CCC[C@H]3C)cc2)CC1]

is a high affinity and highly selective histamine H3 receptor ligand. This compound can be obtained by the evaporation of the dichloromethane solution of the resulting product prepared in Example 11 of WO 2014/136075 or by base liberation after the isolation of the hydrochloride salt—which is obviously known by one skilled in the art.

In vitro the compound behaves functionally as a receptor antagonist/inverse agonist and it also shows histamine H3 receptor antagonism in vivo in rats.

ASD is a complex, very challenging and prevalent neurodevelopmental condition that affects approximately 1% of both children and adults (Brugha et al., Arch. Gen. Psychiatry. 2011, 68:459-465; Murphy et al., Neuropsychiatr. Dis. Treat. 2016, 12:1669-1686). The disorder is characterized by the two core symptoms of

1) socio-communicational dysfunctions (persistent deficits in social communication and social interaction across multiple contexts) as well as

2) restricted (repetitive, stereotyped) behaviors and thinking (restricted, repetitive patterns of behavior, interests, or activities)

### (Diagnostic and Statistical Manual of Mental Disorders, Fifth Edition, Pp. 50-59).

Social impairments comprise abnormal social approach, failure of normal back-and-forth communication, failure to initiate and reciprocate interaction. Communicational deficits may include poorly integrated verbal and nonverbal communication, abnormal eye contact and body language, deficits in understanding gestures, lack of facial expressions. In general, deficits in developing, maintaining and understanding relationships, adjusting to social situations, sharing imaginative play and absence of interest in peers may be present. With respect to the other core symptom domain, stereotyped or repetitive motor movements, insistence on sameness and routines, highly fixated interests that are abnormal in intensity or focus and abnormal sensory reactivity can be identified.

In addition to the core symptoms, ASD is also often accompanied by associated or comorbid symptoms including intellectual disability, attention deficit, hyperactivity, mood disorders, seizures, sleep problems, etc. (Lai et al., Lancet 2013, 383(9920):896-910). A further frequently associated symptom domain is irritability that comprises tantrums, aggression towards others, self-injurious behavior and mood swings. Furthermore, ASD is associated with substantial impairments in adaptive behaviour. Symptoms of ASD are present from early childhood and significantly impair everyday, social, occupational, and other important areas of functioning.

Histamine H3 receptor antagonists have been extensively investigated with the aim to develop drugs for the treatment of various diseases such as Alzheimer's disease, obesity, schizophrenia, myocardial ischaemia, migrain, nasal congestion, etc. (Leurs et al., Nat. Rev. Drug Disc. 2005, 4:107-120; Berlin et al., J. Med. Chem. 2011, 54:26-53). A large number of compounds showed promising preclinical results and entered the clinical phase in indications such as excessive daytime sleepiness (EDS) associated with Parkinson disease, EDS secondary to obstructive sleep apnoea, epilepsy, schizophrenia, dementia and attention deficit hyperactivity disorder (Kuhne et al., Exp. Opin. Invest. Drugs 2011, 20:1629-1648). Histamine H3 receptor antagonist/inverse agonists have been considered as potential pharmacotherapeutic agents for the treatment of sleep disorders (Barbier and Bradbury, CNS Neurol. Disord. Drug Targets 2007, 6:31-43). So far however, only one histamine H3 receptor antagonist, the first-in-class pitolisant (under the brand name of Wakix), has received market authorization from the European Medicines Agency to treat narcolepsy with or without cataplexy in adults (Kollb-Sielecka et al. Sleep Med. 2017, 33:125-129). Notably, there is no drug with a selective histamine H3 receptor antagonist mechanism of action in clinical development or on the market to treat symptoms of ASD.

Baronio and co-workers reported that the histamine H3 receptor antagonist ciproxifan administered at 3 mg/kg intraperitoneally improved social deficits and excessive repetitive behavior in the prenatal valproate model of ASD in mice (Baronio et al., PLOS One 2015, 10:1). In the same model, ciproxifan did not have an effect on social novelty preference, a cognitive function impaired in prenatally VPA-exposed mice. Ciproxifan is a non-selective antagonist of the histamine H3 receptor. Beside its antagonism at the histamine H3 receptor, ciproxifan also inhibits human and rat monoamine oxidase A and B in the micromolar concentration range with a slight preference for monoamine oxidase B (Hagenow et al., Sci. Rep. 2017 7:40541). Ciproxifan given at 3 mg/kg intraperitoneally produces plasma exposures in the micromolar range (Ligneau et al., J. Pharm. Exp. Ther. 1998, 287:658-666). Therefore, the behavioral effects reported by Baronio et al. (2015) are due either to monoamine oxidase inhibition or to the combination of monoamine oxidase and histamine H3 receptor antagonism.

Prenatal exposure to valproate (valproic acid, VPA) in rodents is a widely accepted preclinical model of autism spectrum disorder. Prenatal valproate exposure is known to increase the risk of ASD in humans (Christensen et al, JAMA 2013, 309) and the presence of VPA during intrauterine life in rodents is also known to lead to an autistic-like phenotype in the offspring (Roullet et al, Neurotox. Teratol. 2013, 36, 45-56). VPA in rodents is administered typically around the 12th day of embryonic development when the closure of the neural tube as well as establishment of the cranial nerve nuclei and the cerebellum occurs. The histone deacetylase inhibitory effect of VPA interferes with the above mentioned developmental steps and thereby brings about the autistic-like phenotype in the offspring (Kataoka et al., Int. J. Neuropsychopharm. 2011 16:91-103). The autistic-like phenotype comprises—not exclusively—impaired communication in rat pups, impaired social play behavior in adolescent rats and defective sociability in the 3-chamber assay in adult rats. Since the physiological origin and the symptoms of the prenatal VPA model show a good match with the human condition, this is a generally accepted rodent model of ASD with high translational value.

The unmet medical need in ASD is enormous, since there is no pharmacological treatment currently available for the treatment of core symptoms in ASD. While there is no approved drug for the treatment of core symptoms (socio-communicational dysfunctions and restricted and repetitive behaviours), only two antipsychotics of the many available drugs of the same class—risperidone and aripiprazole—have been approved by the US Food & Drug Administration for the treatment of ASD-associated irritability in children, ages 5-16 years (risperidone) or 6-17 years (aripiprazole). Aripiprazole has also been approved for this purpose in Japan. Although large efforts have been put into clinical research, no effective pharmacological treatment has been identified until now to alleviate the core symptom domains of ASD.

### SUMMARY OF THE INVENTION

The present invention relates to Compound of Formula 1, its pharmaceutically acceptable salts, derivatives, pharmaceutical compositions, metabolites and combinations for use in the treatment of symptoms of autism spectrum disorder.

### BRIEF DESCRIPTION OF THE FIGURES

FIG. 1. Repeated (once daily for 8 days) per os administration of 1-[4-(4-{3-[(2R)-2-methylpyrrolidin-1-yl]-propoxy}-phenoxy)-piperidin-1-yl]-ethan-1-on dicitrate salt (CMPD) reversed the social play deficit induced by prenatal valproate treatment on postnatal day 30.

FIG. 2. Repeated (once daily for 8 days) per os administration of 1-[4-(4-{3-[(2R)-2-methylpyrrolidin-1-yl]-propoxy}-phenoxy)-piperidin-1-yl]-ethan-1-on dicitrate salt reversed the social preference deficit induced by prenatal valproate treatment on postnatal day 59.

FIG. 3. Repeated (once daily for 9 days) per os administration of 1-[4-(4-{3-[(2R)-2-methylpyrrolidin-1-yl]-propoxy}-phenoxy)-piperidin-1-yl]-ethan-1-on dicitrate salt reversed the social recognition deficit induced by prenatal valproate treatment on postnatal day 60.

### DETAILED DESCRIPTION OF THE INVENTION

In order to assess the potential of Compound of Formula 1, it was investigated in the prenatal valproate model of ASD. Surprisingly, it has been found that Compound of Formula 1, showed great benefit in said animal model that recapitulates the symptoms of ASD. As described in the Examples, the compound was able to reverse behavioral deficits in rats that had been exposed to valproate during their intrauterine life. These results indicate that Compound of Formula 1 and its derivatives are of therapeutic use against the symptoms of ASD in human patients.

Accordingly, the present invention relates to Compound of Formula 1 and/or its derivatives and/or its metabolites and/or pharmaceutically acceptable salts thereof for use in the treatment of symptoms of ASD.

In another preferred embodiment, the present invention is directed to Compound of Formula 1 or pharmaceutically acceptable salts thereof for use in the treatment of symptoms of ASD.

In another preferred embodiment, the present invention directed to Compound of Formula 1 and/or its derivatives and/or its metabolites and/or pharmaceutically acceptable salts thereof for use in the treatment of socio-communicational dysfunctions as core symptom of ASD.

In another preferred embodiment, the present invention directed to Compound of Formula 1 and/or its derivatives and/or its metabolites and/or pharmaceutically acceptable salts thereof for use in the treatment of restricted and repetitive behaviours as core symptom of ASD.

In another preferred embodiment, the present invention directed to Compound of Formula 1 and/or its derivatives and/or its metabolites and/or pharmaceutically acceptable salts thereof for use in the treatment of irritability associated with ASD.

The present invention also relates to a pharmaceutical composition comprising Compound of Formula 1 and/or its derivatives and/or its metabolites and/or pharmaceutically acceptable salts thereof and pharmaceutically acceptable excipients, for use in the treatment of symptoms of ASD.

The present invention also relates to a pharmaceutical composition comprising Compound of Formula 1 and/or its derivatives and/or its metabolites and/or pharmaceutically acceptable salts thereof and pharmaceutically acceptable excipients, for use in the treatment of core symptoms of ASD.

In another preferred embodiment the invention relates to a pharmaceutical composition for use as defined above, wherein the core symptom of ASD is socio-communicational dysfunctions.

In another preferred embodiment the invention relates to a pharmaceutical composition for use as defined above, wherein the core symptom of ASD is restricted and repetitive behaviours.

In another preferred embodiment the invention relates to a pharmaceutical composition for use as defined above, wherein the condition to be treated is irritability associated with ASD.

The present invention also relates to the method of treating ASD comprising administering to a subject in need a therapeutically effective amount of a Compound of Formula 1 and/or its derivatives and/or its metabolites and/or pharmaceutically acceptable salts thereof.

The present invention also relates to the method of treating symptoms of ASD comprising administering to a subject in need a therapeutically effective amount of a Compound of Formula 1 and/or its derivatives and/or its metabolites and/or pharmaceutically acceptable salts thereof.

In another preferred embodiment the invention relates to the method of treating socio-communicational dysfunctions as core symptom of ASD comprising administering to a subject in need a therapeutically effective amount of a Compound of Formula 1 and/or its derivatives and/or its metabolites and/or pharmaceutically acceptable salts thereof.

In another preferred embodiment the invention relates to the method of treating restricted and repetitive behaviours as core symptom of ASD comprising administering to a subject in need a therapeutically effective amount of a Compound of Formula 1 and/or its derivatives and/or its metabolites and/or pharmaceutically acceptable salts thereof.

In another preferred embodiment the invention relates to the method of treating irritability associated with ASD comprising administering to a subject in need a therapeutically effective amount of a Compound of Formula 1 and/or its derivatives and/or its metabolites and/or pharmaceutically acceptable salts thereof.

The present invention also relates to the method of treating symptoms of ASD comprising administering to a subject in need a pharmaceutical composition comprising a therapeutically effective amount of a Compound of Formula 1 and/or its derivatives and/or its metabolites and/or pharmaceutically acceptable salts thereof.

The compositions according to the present invention can be administered by oral, transdermic, parenteral, intranasal and rectal routes. The compositions can especially be administered by the oral route in an appropriate formulation. The dosages of the compound of Formula 1 or its pharmaceutically acceptable salts, derivatives, or metabolites in the compositions of the invention can be adjusted to obtain an amount of active substance that results in the desired therapeutic response. Therefore, the dosage level depends on the desired therapeutic response, the route of administration, the expected duration of treatment and other factors such as age, gender or body weight of the patient. The dosages are from 0.01 to 40 mg daily and can be titrated to effect. The dosages are preferably from 0.01 to 20 mg daily, more preferably from 0.01 to 10 mg daily.

Compound of Formula 1 may also be used in combination with at least one other active ingredient for the treatment of comorbid symptoms of ASD (e.g., psychostimulants, antipsychotics, compounds acting on the oxytocin/vasopressin receptor system, antidepressants, anxiolytics, antihypertensives, antiepileptics, narcotics, spasmolytics or other agents).

The present invention provides pharmaceutical compositions comprising a combination of the compound of Formula 1, as defined above with one or more other active ingredients. The pharmaceutical composition may comprise at least one compound of the invention together with one ore more other active ingredients in a single dosage form or separately. The combinational composition may be administered simultaneously, separately or sequentially.

The present invention relates also to pharmaceutical compositions for use in pediatric use such as, but not limited to, solutions, syrups, elixirs, suspensions, powders for the preparation of suspensions, dispersible or effervescent tablets, chewable tablets, orodispersible tablets, tablets or coated tablets, orally sparkling powders or granules, capsules.

Psychostimulants include, but not limited to, centrally acting sympathomimetics (amphetamine, methylphenidate, modafinil, atomoxetine), nootropics (vinpocetine, donepezil, memantine) or other psychostimulants.

Antipsychotics include, but not limited to, typical and atypical antipsychotics, such as haloperidol, pimozide, clozapine, olanzapine, quetiapine, sertindole, ziprasidone, lurasidone, risperidone, aripiprazole, cariprazine, brexpiprazole, iloperidone, paliperidone, lithium.

Compounds acting on the oxytocin/vasopressin receptor system include, but not limited to, oxytocin, carbetocin, arginine-vasopressin, balovaptan, relcovaptan, conivaptan, selepressin, nelivaptan, tolvaptan, atosiban.

Antidepressants include, but not limited to, non-selective monoamine reuptake inhibitors (desipramine, imipramine, clomipramine, amitriptyline, nortriptyline), serotonin modulator and stimulators (vilazodone, vortioxetine), selective serotonin reuptake inhibitors (fluoxetine, paroxetine, sertraline, fluvoxamine, citalopram, escitalopram), non-hydrazide monoamine oxidase inhibitor (moclobemide) or other agents (mianserin, trazodone, nefazodone, mirtazapine, tianeptine, venlafaxine, milnacipran, reboxetine, duloxetine, agomelatine, bupropion, gepirone).

Anxiolytics include, but not limited to, benzodiazepines (diazepam, chlorodiazepoxide, oxazepam, lorazepam, alprazolam), azaspirode-diones (buspirone).

Antihypertensives include, but not limited to, imidazoline receptor agonists (clonidine, guanfacine) and a combination of these substances with a diuretic.

Antiepileptics include, but not limited to, barbiturates and their derivatives (phenobarbital), hydantoin derivatives (phenytoin), succinimide derivatives (ethosuximide), benzodiazepine derivative clonazepam, carboxamide derivatives (carbamazepine, oxcarbazepine), fatty acid derivatives (valproic acid, valpromide, vigabatrin, tiagabine) and other antiepileptics (lamotrigine, topiramate, gabapentin, levetiracetam, zonisamide, pregabalin).

Narcotics include, but not limited to, barbiturates (pentobarbital), benzodiazepines (midazolam), cyclopyrrolone benzodiazepine derivatives (zopiclone, zolpidem), melatonin receptor agonists (melatonin, ramelteon).

Spasmolytics or antispasmodics include, but not limited to centrally acting agents (baclofen, arbaclofen, tolperisone) and papaverine.

Other agents include, but not limited to, medicinal products (probiotics, digestive aids/digestives, herbal extracts), vitamins (both water soluble and fat soluble, such as, but not limited to, vitamin A, D3, E, K, B1, B5, B6, B12, C or their derivatives) and nutritional supplements (coenzymes eg. Q10, flavonoids eg., resveratrol, lecithin, unsaturated fatty acids, including fatty acids ω3 and ω6).

Accordingly, the present invention also relates to a pharmaceutical composition for use in the treatment of ASD comprising
1) Compound of Formula 1 and/or its derivatives and/or its metabolites and/or pharmaceutically acceptable salts thereof and
2) at least one other active ingredient, which is selected from the group consisting of psychostimulants/nootropics, antipsychotics, antidepressants, anxiolytics, antihypertensives, antiepileptics, narcotics and spasmolytics
   and
3) one or more pharmaceutically acceptable carriers, diluents and excipients.

The present invention also relates to a pharmaceutical composition comprising Compound of Formula 1 and/or its derivatives and/or its metabolites and/or pharmaceutically acceptable salts thereof and at least one other active ingredient for use in the treatment of symptoms of ASD.

In a preferred embodiment the present invention relates to a pharmaceutical combinational composition for use as defined above, wherein the at least one other active ingredient is selected from the group consisting of psychostimulants/nootropics, antipsychotics, antidepressants, anxiolytics, antihypertensives, antiepileptics, narcotics and spasmolytics.

In another preferred embodiment the present invention relates to a pharmaceutical combinational composition for use as defined above, wherein the psychostimulant/nootropic agent is selected from the group comprising amphetamine, methylphenidate, modafinil, atomoxetine, vinpocetine, donepezil and memantine.

In another preferred embodiment the present invention relates to a pharmaceutical combinational composition for use as defined above, wherein the antipsychotic agent is selected from the group comprising haloperidol, pimozide, clozapine, olanzapine, quetiapine, sertindole, ziprasidone, lurasidone, risperidone, aripiprazole, cariprazine, brexpiprazole, ioperidone, paliperidone and lithium.

In another preferred embodiment the present invention relates to a pharmaceutical combinational composition for use as defined above, wherein the compound acting on the oxytocin/vasopressin receptor system is selected from the group comprising oxytocin, carbetocin, arginine-vasopressin, balovaptan, relcovaptan, conivaptan, selepressin, nelivaptan, tolvaptan and atosiban.

In another preferred embodiment the present invention relates to a pharmaceutical combinational composition for use as defined above, wherein the antidepressant agent is selected from the group comprising desipramine, imipramine, clomipramine, amitriptyline, nortriptyline, vilazodone, vortioxetine, fluoxetine, paroxetine, sertraline, fluvoxamine, citalopram, escitalopram moclobemide, mianserin, trazodone, nefazodone, mirtazapine, tianeptine, venlafaxine, milnacipran, reboxetine, duloxetine, agomelatine, bupropion and gepirone.

In another preferred embodiment the present invention relates to a pharmaceutical combinational composition for use as defined above, wherein the anxiolytic agent is selected from the group comprising diazepam, chlorodiazepoxide, oxazepam, lorazepam, alprazolam and buspirone.

In another preferred embodiment the present invention relates to a pharmaceutical combinational composition for use as defined above, wherein the antihypertensive agent is selected from clonidine and guanfacine.

In another preferred embodiment the present invention relates to a pharmaceutical combinational composition for use as defined above, wherein the antiepileptic agent is selected from the group comprising phenobarbital, phenytoin, ethosuximide, clonazepam, carbamazepine, oxcarbazepine, valproic acid, valpromide, vigabatrin, tiagabine, lamotrigine, topiramate, gabapentin, levetiracetam, zonisamide and pregabalin.

In another preferred embodiment the present invention relates to a pharmaceutical combinational composition for use as defined above, wherein the narcotic agent is selected from the group comprising pentobarbital, midazolam, zopiclone, zolpidem, melatonin and ramelteon.

In another preferred embodiment the present invention relates to a pharmaceutical combinational composition for use as defined above, wherein the spasmolytic is selected from the group comprising baclofen, arbaclofen, tolperisone and papaverine.

### Preparation of Pharmaceutical Compositions

The following formulation examples illustrate representative pharmaceutical compositions of this invention. The present invention however is not limited to the following pharmaceutical compositions.

### A) Solid Oral Dosage Forms

Tablets

| | |
|---|---|
| Active substance(s) | 0.005-90% |
| Filler | 1-99.9% |
| Binder | 0-20% |
| Disintegrant | 0-20% |
| Lubricant | 0-10% |
| Other specific excipient(s) | 0-50% |

### B) Parenteral Dosage Forms

Intravenous injections

| | |
|---|---|
| Active substance(s) | 0.001-50% |
| Solvent | 10-99.9% |
| Co-solvent | 0-99.9% |
| Osmotic agent | 0-50% |
| Buffering agent | q.s. |

### C) Other Dosage Forms

Suppositories

| | |
|---|---|
| Active substance(s) | 0.0003-50% |
| Suppository base | 1-99.9% |
| Surface-active agents | 0-20% |
| Lubricant | 0-20% |
| Preservatives | q.s. |

### Definitions

The term “active ingredient” means Compound of Formula 1, its pharmaceutically acceptable salts, active metabolites and derivatives.

The term “active metabolite” means such metabolites produced by different routes of biotransformation whose biological activity is similar to that of the parent compound.

The term “affinity” means the attraction of a drug for a biological target; it is a chemical term used to quantify the strength of drug-target interaction.

The term “antagonist” means a compound that associates with a receptor and produces no response or prevents the response generated by an agonist of the same receptor.

The term “derivative” means such compounds that are produced by chemical modification of the compound of the invention resulting not exclusively in prodrugs, deuterated compounds, etc.

The term “excipient” defines a chemical compound that facilitates the incorporation of a compound into cells or tissues. The excipients applicable in the preparation may be selected from the following categories, such as, but not limited to, fillers of tablets and capsules, binders of tablets and capsules, modified drug release agents, disintegrants, glidants, lubricants, sweeteners, taste-masking agents, flavorants, coating materials, surfactants, stabilizers, preservatives or antioxidants, buffering agents, complexing agents, wetting or emulsifying agents, salts for adjusting the osmotic pressure, lyophilization excipients, microencapsulating agents, ointment materials, penetration enhancers, solubilizers, solvents, suppository materials, suspending agents. Suitable pharmaceutical excipients can be for example: starch, microcrystalline cellulose, talc, glucose, lactose, gelatin, silica, talc, magnesium stearate, sodium stearate, glycerol monostearate, cellulose derivatives, sodium chloride, glycerol, propylene glycol, water, ethanol and the like.

The term “inverse agonist” means an agent that binds to and decreases the activity of constitutively active receptors thus induces pharmacological response opposite to the agonist.

The term “patient” refers to a human who received an ASD diagnosis.

The term “pharmaceutically acceptable” describes an ingredient that is useful in preparing a pharmaceutical composition and is generally safe, non-toxic and neither biologically nor otherwise undesirable, and includes those acceptable for human pharmaceutical use.

The term “pharmaceutical composition” refers to a mixture of a compound of the invention with other chemical components, such as pharmaceutically acceptable excipients e.g. diluents or carriers. The pharmaceutical composition facilitates administration of the compound to the subject.

The term “pharmaceutically acceptable salt” refers to a conventional acid addition salt which preserves the biological efficacy and properties of the compounds of formula (I) and which can be formed with suitable non-toxic organic or inorganic acids. Examples of acid addition salts include salts derived from inorganic acids, such as, but not limited to, (mono- or di-) hydrochloric acid, (mono- or di-) hydrobromic acid, hydroiodic acid, sulfuric acid, sulphamic acid, phosphoric acid, nitric acid and perchloric acid and derived from various organic acids, such as, but not limited to, acetic acid, propionic acid, benzoic acid, glycolic acid, phenylacetic acid, salicylic acid, malonic acid, maleic acid, oleic acid, pamoic acid, palmitic acid, benzenesulfonic acid, toluenesulfonic acid, methanesulfonic acid, oxalic acid, tartaric acid, succinic acid, (mono- or di-) citric acid, malic acid, lactic acid, glutamic acid, fumaric acid and the like. These salts often exhibit more favorable solubility properties than the compounds used for their preparation and are therefore more suitable for use in the preparation of various pharmaceutical formulations.

The term “selective” means that a ligand binds satisfactorily more preferentially to one molecular target than other targets which eventually may result in different pharmacological activity.

As used herein, the term “treatment” refers to the alleviation of a specific pathological condition, the elimination or reduction of one or more of the symptoms of the condition, the slowing or elimination of the progression of the disease state, and the prevention or delay of recurrency of the pathological condition of a patient or subject already suffering from or diagnosed with the disease.

### The Prenatal Valproate Model

As described in the background, the prenatal valproate (VPA) model has excellent construct and face validity, therefore it is a widely accepted disease model of ASD. Since there is no approved drug on the market for the treatment of the core symptoms of ASD, predictive validity of the model can be only assessed on the basis of compounds that showed efficacy signals in humans. Compounds that produced efficacy signals in ASD subjects comprise eg., oxytocin (Andari et al., PNAS 2010, 107:4389-4394). Oxytocin has been found to improve behavioral impairments in the prenatal VPA model (Hara et al., Horm. Behav. 2017, 96:130-137), therefore it can be assumed that the model may be able to predict usefulness in the treatment of core ASD symptoms in patients.

In this method, time-mated female Wistar rats are administered a single dose of VPA (600 mg/kg, i.p.) on gestational day 12.5. Offspring are housed according to standard laboratory conditions until time of behavioral testing. Animals are housed in groups of 4 in conventional cages and maintained at 22-24° C. on a standard 12 hours light/dark cycle, with food and water available ad libitum. After investigational drug treatment, offspring are examined behaviorally in tests relevant for the assessment of autistic behavior. These tests include social play, social preference and social recognition memory. These tests are suitable to assess behavioral elements that represent core ASD symptoms in experimental animals. Efficaciousness of an investigational compound (ie. improvement of behavioral deficits induced by prenatal VPA exposure) may indicate usefulness in the pharmacotherapy of core symptoms of ASD. Doses of the investigational drug were corrected for the dicitrate salt with a correction factor of 2.07 and are expressed as free base. Reference is herein made to a parallel patent application filed by the applicant with the title of “Selective histamine H3 receptor antagonist acid addition salts and process for the preparation thereof” wherein the salts, including dicitrate salt is disclosed in detail.

Maternal deprivation-induced ultrasonic vocalization in rat pups is a readout of socio-communicational function that is impaired in offspring after prenatal exposure to VPA (Gandal et al., Biol. Psychiatry 2010, 68, 1100-1106). To induce ultrasonic calls, prenatally VPA treated rat pups are placed individually into a cage where calls are being recorded with bat microphones. Calls are digitized with an audio filter and ultrasonic vocalization is recorded and quantified with SonoTrack software (Metris bv. The Netherlands). Baseline vocalization is measured on postnatal days 11-12 days for 10 min. Animals are divided into homogenous groups based on baseline vocalizations. On postnatal day 13, animals are treated p.o. with the appropriate doses of drug or vehicle 60 min before measurement and then returned to their nests until recording. Ultrasonic call counts are recorded for 10 min. Statistical analysis is performed on ultrasonic call counts with the non-parametric Kruskal-Wallis test and the post hoc Dunnett test.

Social play is a type of social interaction that is highly typical of adolescent mammals including rodents as well as humans (Vanderschuren and Trezza, Curr. Topics Behav. Neurosci. 2014, 16:189-212). Social play behavior is indicative of healthy social functioning in adult life and is defective after prenatal VPA treatment in the offspring (Schneider and Przewlocki Neuropsychopharmacology 2005, 30:80-89). At postnatal day 30, following 8 days treatment with the test compound, prenatally VPA-treated rats are evaluated for juvenile play behavior. The test is carried out in a test arena unknown to the rats with pairs of animals from the same treatment group over a 15 min trial. Animals are scored for duration of social play behavior. Statistical analysis is performed by using one-way ANOVA and Student's t test.

The social preference and recognition memory assays in a 3-chamber apparatus are further indicators of intact social behavior in rats. The preference of a conspecific over an inanimate object as well as the ability to distinguish between familiar and novel conspecifics are necessary for normal social functioning and are defective in the prenatal VPA model in rats (Bambini-Junior et al., Brain Res. 2011, 1408:8-16). Social preference and social recognition memory are investigated in a 3-chamber apparatus. On postnatal day 59, prenatally VPA-treated rats are assessed for their social preference following 8 days of per os treatment with the test compound. On postnatal day 60, the same rats are assessed for their social recognition memory after 9 days of per os treatment with the test compound. Statistical analysis is performed by using two-way ANOVA and Student's t test.

### DETAILED DESCRIPTION OF THE FIGURES

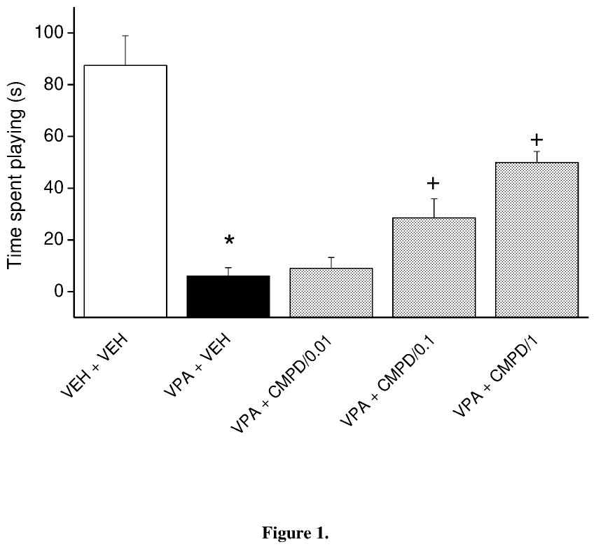

FIG. 1. Repeated (once daily for 8 days) per os administration of 1-[4-(4-{3-[(2R)-2-methylpyrrolidin-1-yl]-propoxy}-phenoxy)-piperidin-1-yl]-ethan-1-on dicitrate salt (CMPD) reversed the social play deficit induced by prenatal valproate treatment on postnatal day 30 (*P<0.05 Student's t-test versus “prenatal VPA+vehicle treatment” (VPA+VEH)). The effect of 1-[4-(4-{3-[(2R)-2-methylpyrrolidin-1-yl]-propoxy}-phenoxy)-piperidin-1-yl]-ethan-1-on dicitrate was statistically significant at the doses of 0.1 and 1 mg/kg (+p<0.05 Bonferroni post hoc test versus “VPA+VEH”). VEH+VEH means prenatal vehicle treatment combined with vehicle treatment before behavioral testing. VPA+VEH animals received prenatal VPA combined with vehicle treatment before behavioral testing. VPA+CMPD/0.01, CMPD/0.1 and CMPD/1 groups received prenatal VPA followed by 0.01, 0.1 and 1 mg/kg 1-[4-(4-{3-[(2R)-2-methylpyrrolidin-1-yl]-propoxy}-phenoxy)-piperidin-1-yl]-ethan-1-on dicitrate, respectively.

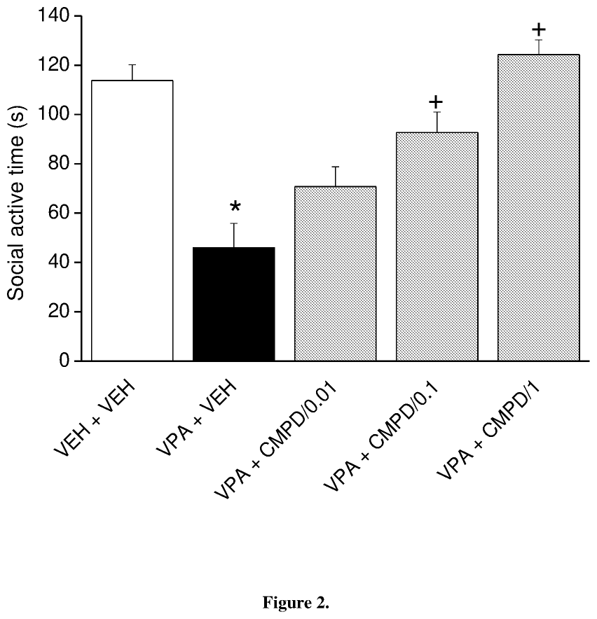

FIG. 2. Repeated (once daily for 8 days) per os administration of 1-[4-(4-{3-[(2R)-2-methylpyrrolidin-1-yl]-propoxy}-phenoxy)-piperidin-1-yl]-ethan-1-on dicitrate salt reversed the social preference deficit induced by prenatal valproate treatment on postnatal day 59 (*P<0.05 Student's t-test versus “prenatal VPA+vehicle treatment” (VPA+VEH). The effect of 1-[4-(4-{3-[(2R)-2-methylpyrrolidin-1-yl]-propoxy}-phenoxy)-piperidin-1-yl]-ethan-1-on dicitrate was statistically significant at the doses of 0.1 and 1 mg/kg (+p<0.05 Bonferroni post hoc test versus “VPA+VEH”). VEH+VEH means prenatal vehicle treatment combined with vehicle treatment before behavioral testing. VPA+VEH animals received prenatal VPA combined with vehicle treatment before behavioral testing. VPA+CMPD/0.01, CMPD/0.1 and CMPD/1 groups received prenatal VPA followed by 0.01, 0.1 and 1 mg/kg 1-[4-(4-{3-[(2R)-2-methylpyrrolidin-1-yl]-propoxy}-phenoxy)-piperidin-1-yl]-ethan-1-on dicitrate, respectively.

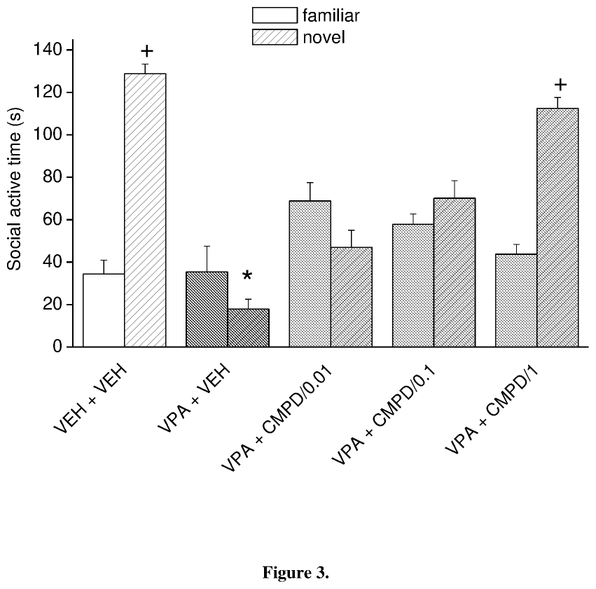

FIG. 3. Repeated (once daily for 9 days) per os administration of 1-[4-(4-{3-[(2R)-2-methylpyrrolidin-1-yl]-propoxy}-phenoxy)-piperidin-1-yl]-ethan-1-on dicitrate salt reversed the social recognition deficit induced by prenatal valproate treatment on postnatal day 60 (*P<0.05 Student's t-test; “novel” versus “familiar”). “Familiar” denotes the time spent investigating a conspecific already known to the test animal, “novel” denotes the time spent investigating a conspecific that is yet unknown to the test animal The effect of 1-[4-(4-{3-[(2R)-2-methylpyrrolidin-1-yl]-propoxy}-phenoxy)-piperidin-1-yl]-ethan-1-on dicitrate was statistically significant at the dose of 1 mg/kg (+p<0.05 Bonferroni post hoc test versus “prenatal VPA+vehicle treatment” (VPA+VEH) familiar). VEH+VEH means prenatal vehicle treatment combined with vehicle treatment before behavioral testing. VPA+VEH animals received prenatal VPA combined with vehicle treatment before behavioral testing. VPA+CMPD/0.01, CMPD/0.1 and CMPD/1 groups received prenatal VPA followed by 0.01, 0.1 and 1 mg/kg 1-[4-(4-{3-[(2R)-2-methylpyrrolidin-1-yl]-propoxy}-phenoxy)-piperidin-1-yl]-ethan-1-on dicitrate, respectively.

The following examples illustrate the invention without limiting the scope thereof.

### Example 1

1-[4-(4-{3-[(2R)-2-methylpyrrolidin-1-yl]-propoxy}-phenoxy)-piperidin-1-yl]-ethan-1-on dicitrate was tested in the social play assay in prenatally valproate-treated rats (FIG. 1). The effect of subacute (once daily for 8 days) administration of 1-[4-(4-{3-[(2R)-2-methylpyrrolidin-1-yl]-propoxy}-phenoxy)-piperidin-1-yl]-ethan-1-on dicitrate on rat social play behavior is shown in FIG. 1. The data represented are mean f SEM of 4 pairs of adolescent (at postnatal day 30) male rats for each group. 1-[4-(4-{3-[(2R)-2-methylpyrrolidin-1-yl]-propoxy}-phenoxy)-piperidin-1-yl]-ethan-1-on dicitrate given orally partially reversed social play deficits in valproate-treated offspring reaching significance at the dose 0.1 mg/kg. Thus, the compound of the invention was able to reduce the social deficit induced by prenatal valproate treatment.

### Example 2

1-[4-(4-{3-[(2R)-2-methylpyrrolidin-1-yl]-propoxy}-phenoxy)-piperidin-1-yl]-ethan-1-on dicitrate was tested on social preference in the 3-chamber apparatus in prenatally valproate-treated rats (FIG. 2). The effect of subacute (once daily for 8 days) oral administration of 1-[4-(4-{3-[(2R)-2-methylpyrrolidin-1-yl]-propoxy}-phenoxy)-piperidin-1-yl]-ethan-1-on dicitrate on social preference is shown in FIG. 2. The data represented are mean f SEM of 8 male rats for each group (at postnatal day 59). 1-[4-(4-{3-[(2R)-2-methylpyrrolidin-1-yl]-propoxy}-phenoxy)-piperidin-1-yl]-ethan-1-on dicitrate given orally fully reversed social preference deficits in valproate-treated offspring at the dose of 1 mg/kg. Thus, the compound of the invention was able to reduce the social deficit induced by prenatal valproate treatment.

### Example 3

1-[4-(4-{3-[(2R)-2-methylpyrrolidin-1-yl]-propoxy}-phenoxy)-piperidin-1-yl]-ethan-1-on dicitrate was tested on social recognition memory in the 3-chamber apparatus in prenatally valproate-treated rats (FIG. 3). The effect of subacute (once daily for 9 days) oral administration of 1-[4-(4-{3-[(2R)-2-methylpyrrolidin-1-yl]-propoxy}-phenoxy)-piperidin-1-yl]-ethan-1-on dicitrate on social recognition memory is shown in FIG. 3. The data represented are mean f SEM of 8 male rats for each group (at postnatal day 60). 1-[4-(4-{3-[(2R)-2-methylpyrrolidin-1-yl]-propoxy}-phenoxy)-piperidin-1-yl]-ethan-1-on dicitrate given orally fully reversed social recognition memory deficits in valproate-treated offspring at the dose of 1 mg/kg. Thus, the compound of the invention was able to reduce the social deficit induced by prenatal valproate treatment.

### Example 4

The behavioral effect of the co-administration of 1-[4-(4-{3-[(2R)-2-methyl-pyrrolidin-1-yl]-propoxy)-phenoxy)-piperidin-1-yl]-ethan-1-one dicitrate and risperidone was studied in prenatally valproate-exposed (300 mg/kg) rat pups on ultrasound emissions induced by separation from their mothers. Risperidone was chosen because of its approval (FDA, US) for the treatment of irritability associated with ASD and its frequent use in the everyday medical practice in ASD (McClellan et al., Curr. Treat: Options Psych. 2016 3:161-181). Furthermore, the dose of risperidone applied in the present study was reported to produce an efficacy signal in the prenatal valproate exposure model (Kuo and Liu, FASEB J 2017, 31(10):4458-4471). Co-administration of oral doses of 1-[4-(4-{3-[(2R)-2-methyl-pyrrolidin-1-yl]-propoxy}-phenoxy)-piperidin-1-yl]-ethan-1-one dicitrate in the dose range of 0.01-1 mg/kg with orally given risperidone (0.003 mg/kg) dose-proportionally reversed the socio-communicational defect induced by prenatal valproate exposure in rat pups.

1-16. (canceled)

17. A method of treating Autism Spectrum Disorder (ASD) or a symptom thereof comprising administering to a patient in need thereof a therapeutically effective amount of compound of Formula 1 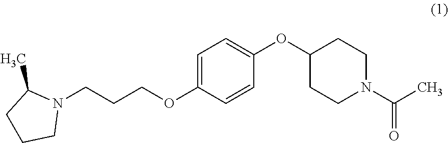 [SMILES: CC(=O)N1CCC(Oc2ccc(OCCCN3CCC[C@H]3C)cc2)CC1]
or a pharmaceutically acceptable salt thereof.

18. The method of claim 17 for treating a symptom of ASD comprising administering to a patient in need thereof a therapeutically effective amount of compound of Formula 1 or a pharmaceutically acceptable salt thereof.

19. The method according to claim 18 wherein the symptom is a core symptom of ASD.

20. The method according to claim 19 wherein the core symptom is a socio-communicational dysfunction.

21. The method according to claim 19 wherein the core symptom is restricted and repetitive behaviours.

22. The method according to claim 18 wherein the symptom is irritability.

23. The method according to claim 17 wherein the pharmaceutically acceptable salt is selected from the group consisting of dihydrobromide, sulphate, oxalate, monocitrate and dicitrate salts.

24. The method according to claim 17 wherein the therapeutically effective amount of said compound is in the range of 0.01-40 mg/day.

25. A pharmaceutical composition for treatment of Autism Spectrum Disorder (ASD) or a symptom of ASD, said pharmaceutical composition comprising a compound of Formula 1 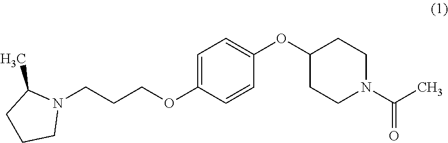 [SMILES: CC(=O)N1CCC(Oc2ccc(OCCCN3CCC[C@H]3C)cc2)CC1]
or a pharmaceutically acceptable salt thereof, and one or more pharmaceutically acceptable ingredient(s).

26. The pharmaceutical composition according to claim 25, wherein the pharmaceutically acceptable salt is selected from the group consisting of dihydrobromide, sulphate, oxalate, monocitrate and dicitrate salts.

27. The pharmaceutical composition according to any one of claim 25, wherein the composition further comprises at least one other active ingredient.

28. The pharmaceutical composition according to claim 25, in the form of a daily dosage comprising an amount of said compound in the range of 0.01-40 mg/day.

29. The pharmaceutical composition according to claim 25 for use in the treatment of ASD.

30. The pharmaceutical composition according to claim 25 for use in the treatment of symptoms of ASD.

31. The pharmaceutical composition of claim 25 comprising at least one other active ingredient selected from the group consisting of psychostimulants, antipsychotics, antidepressants, anxiolytics, antihypertensitives, antiepileptics, narcotics and spasmolytics or antispasmodiscs.

32. The pharmaceutical composition according to claim 31 for use in the treatment of ASD.

33. The pharmaceutical composition according to claim 31 for use in the treatment of symptoms of ASD.

34. The pharmaceutical composition according to claim 25 wherein the one or more pharmaceutically acceptable ingredient(s) is/are selected from the group consisting of carriers, diluents and excipients.
